(19) United States
(12) Patent Application Publication
(10) Pub. No.: US 2011/0238090 A1
(43) Pub. Date: Sep. 29, 2011

(54) METHODS AND DEVICES FOR DELIVERING SUTURES IN TISSUE

(75) Inventor: Scott Hyler HENEVELD, Whitmore, CA (US)

(73) Assignee: Spirx Closure, LLC, Whitmore, CA (US)

(21) Appl. No.: 13/011,787

(22) Filed: Jan. 21, 2011

Related U.S. Application Data
(63) Continuation of application No. PCT/US2009/051442, filed on Jul. 22, 2009.
(60) Provisional application No. 61/135,479, filed on Jul. 22, 2008.

(51) Int. Cl. A61B 17/04

(52) U.S. Cl. 606/144

(57) ABSTRACT

Methods and devices for driving a suture assembly employing elastically pre-shaped needles for piercing a tissue. The pre-shaped needles are held in a constrained state and can revert to a natural pre-shaped state prior to or during ejection from the device before entry into tissue allowing for the suture to follow a defined path similar to the pre-shaped needle such that removal of the needle allows for subsequent securing of the suture in or around tissue.

### CROSS-REFERENCE TO RELATED APPLICATIONS

This application is a continuation of PCT International Patent Application No. PCT/US2009/051442 filed Jul. 22, 2009 which claims the benefit of priority to U.S. Provisional Application No. 61/135,479 filed Jul. 22, 2008, each of which is incorporated by reference herein in its entirety.

### BACKGROUND OF THE INVENTION

The present invention relates generally to systems and methods for the driving of a needle or suture through or into body tissue (typically, the needle will be affixed to a suture that remains in the tissue) using a catheter, introducer or other minimally invasive means. The methods and devices described herein can be used in any number of medical procedures, including but not limited to, approximating tissue (e.g., bring separated tissue together), ligating tissue (e.g., encircling or tying off), and fixating of tissue (attaching tissue to another structure or different tissue).

Commonly known suture systems mechanically drive needles thru the tissue wall to create passage for a suture. Such mechanisms are often complicated and require a skilled operator. In addition, the conventional mechanisms can involve many procedural steps to manipulate a needle to conform to a path to properly position the suture into tissue.

Conventional suture driving systems used for wound closure provides one example of existing suture driving systems. Such wound closure systems are used in transluminal medical procedures that are seeing a rise in popularity due to the reduction in surgical damage to healthy tissue, decreased recovery time, and ultimate cost savings to the patient associated with these procedures.

These transluminal procedures typically require a puncture into a body lumen and through the overlying tissue for the passing of catheters, guide wires, laparoscopes, endoscopes, vascular devices, etc. as required by the particular procedure. The punctures are created with instruments such as access needles, trocar, introducer sheaths, or other access devices and may measure from 1 to upwards of 15 mm in diameter. After completion of the procedure, the physician can utilize a closure system to close the puncture quickly to prevent further bleeding.

Manual compression of arterial or venous punctures is a common closure technique and an alternative to such closure systems. In this closure technique, medical personnel apply continuous pressure to the wound site allowing the blood to eventually clot sufficiently sealing the wound. However, this technique is typically very time consuming, requires the patient to bedridden for an extended time, and is not applicable for punctures over 4 mm. The longer recovery time increases overall cost and decreases patient satisfaction.

Sutures remain the preferred method of sealing such wounds, but the limited access and small size of the typical wound formed during a transluminal procedure complicates the task of sealing these wounds.

Generally, a physician must introduce a suture needle through the tissue tract and into the body lumen, position the needle; then passed the needle through tissue pulling the suture through as well. A number of devices are disclosed in U.S. Pat. No. 5,374,275 to Bradley et al., U.S. Pat. No. 5,364,408 to Gordon, U.S. Pat. No. 5,320,632 to Heidmueller, U.S. Pat. No. 5,403,329 to Hinchcliffe, U.S. Pat. No. 5,368,601 to Sauer et al., U.S. Pat. No. 5,431,666 to Auer et al. and international publications WO 94/13211 and WO 95/13021 each of the above references is incorporated by reference herein.

While these devices allow for sealing of the wound and driving the suture and needle through tissue, they are relatively complex and employ a significant number of moving parts. Accordingly, these devices are relatively costly to produce and are prone to mechanical failure.

U.S. Pat. Nos. 5,527,322, 5,792,152, 6,206,893, and 6,517,553 all to Klein U.S. and U.S. Pat. No. 5,972,005 to Stalker (each of the above references is incorporated by reference herein) describes devices employing flexible or pre-shaped curved needles that are deformed from a natural shape during insertion or during advancement in tissue to close a puncture wound. U.S. Pat. No. 7,377,926 to Topper et al. (incorporated by reference herein) teaches another system for inserting a needle. In this variation, the insertion device houses a bendable needle in one of the jaws and is adapted to carry a suture

However, systems, such as those described above often deform a needle to drive a suture. Deformation of the needle in this manner often results in device malfunction when placing the suture, or requires significant additional complex components to ensure proper movement of the needle and suture as desired. Accordingly, there remains a need for a simple mechanized device and method to accurately and precisely drive a suture through tissue in a constrained space such as is required in less invasive procedures.

Such systems can also perform closure of openings in organs, whether to repair a defect, to close a wound, or to close an incision made in the organ for the purpose of accessing the organ to perform a separate medical procedure. As one example, when performing valve repair or replacement within the heart, a surgeon can access an apex of the heart after performing a thoracotomy or a mini-thoracotomy. The thoracotomy allows the surgeon to manually close the opening in the heart tissue via a suture pattern. Such a pattern can include one or more concentric purse string suture patterns to ensure closure of the opening into the heart. Percutaneous access to the heart to perform such valve procedures results in many of the same benefits as other percutaneous procedures. Namely, reduced complications, cost and recovery time on the part of the patient. However, percutaneous access leaves the surgeon with a small access path to close the opening in the heart.

The anatomical structure of the apical area permits the introduction of various surgical devices and tools into the heart without significant disruption of the natural mechanical and electrical heart function. Access to the heart through the femoral vessels in percutaneous methods is limited to the diameter of the vessel (approximately 8 mm). However, access to the heart through the apical area allows for a significantly larger access path (approximately 25 mm). Thus, apical access to the heart permits greater flexibility with respect to the types of devices and surgical methods that may be performed in the heart and great vessels. Such access is disclosed in Bergheim US Patent Application 20050240200, the entirety of which is incorporated by reference. Accordingly, there remains a need for a simple mechanized device that can accurately and precisely drive a suture through tissue allows the surgeon to close the heart tissue and complete the procedure in a percutaneous manner.

In addition, the methods and systems described herein have additional uses other than closure of tissue. In another example, U.S. Patent Application No. 20070203479 to Auth et al. (incorporated by reference herein) describes methods and devices, and systems for the partial or complete closure or occlusion of a patent foramen ovale (“PFO”). An improved suture driving device can be used for fixating tissue and eliminate the need for such implantable devices.

Accordingly, the need continues to exist for an improved suturing systems and methods that drive a suture for approximating tissue, ligating tissue, and/or fixating of tissue.

### SUMMARY OF THE INVENTION

The following description includes an example of the methods and devices within the scope of this disclosure. It is also contemplated that combinations of aspects of various embodiments as well as the combination of the various embodiments themselves is within the scope of this disclosure.

In one variation, the invention includes a suture driving assembly for positioning a suture in a tissue section, the assembly comprising at least one needle assembly having a tissue piercing end distal to an elongate shaped section, the elongate shaped section having a curvilinear shape, the elongate shaped section being elastically deformable when restrained into a strained state and upon release assumes the curvilinear shape, the suture coupled to the needle assembly; a main body having a tissue engaging surface at a distal end, at least one constraining channel and at least one retrieving channel each of which having an opening at the tissue engaging surface; such that when the elongate shaped section of the needle assembly is in the restraining portion, the elongate shaped section is deformed into the strained state and when the elongate shaped section advances through the guide segment portion, the elongate shaped section assumes the curvilinear shape, upon continued advancement the elongated shaped section exits through the opening of constraining channel in the curvilinear shape; a suture retriever assembly located in the needle retrieving channel.

The suture driving assemblies described herein can optionally include an expandable member axially moveable relative to the tissue engaging surface, the expandable member having a first reduced profile and an expanded profile, where in the reduced profile the expandable member can advance through an opening in the tissue section and where the expandable member can be withdrawn toward the tissue supporting face to secure the tissue section therebetween.

The needle assembly as well as the number of needle assemblies can vary depending upon the type of suture stitch required. For example, the device can include a single needle assembly having a single shaped section or multiple shaped sections. In alternate variations, the assembly comprises two or more needle assemblies. The needle assemblies as well as the shaped portions used in any particular suture driving mechanism need not have the same shape. Instead, a single suture driving assembly can use needle assemblies of differing shapes at the same time. However, the spacing and relation of the constraining channel and the retrieval channel shall be adjusted to accommodate a particular shape and configuration of a particular needle assembly.

In certain variations, the constraining channel can include a first cross-sectional shape and the guide segment has a second cross sectional shape, where the first and second cross-sectional shapes are different, where the second cross sectional shape permits at least a part of the shaped section of the needle assembly entering the guide segment to revert to the curvilinear shape prior to entry into the tissue.

The sutures used in the devices and methods described herein can include a needle assembly comprising a needle lumen extending through at least the tissue piercing end and where the suture is removably nested within the needle lumen. In additional variations, the suture can be located exterior to the needle assembly so that a first free end of the suture is inserted into the needle lumen at the tissue piercing end. In another variation, a single suture can be affixed at both ends to a needle assembly where the needle assembly comprises two shaped sections with each having a tissue piercing end.

Sutures used in the present devices and methods can be front loaded into a needle assembly. As a result, a suture retriever assembly can remove the suture from the needle assembly via a front portion of the needle assembly. In one example, the suture retriever assembly comprises at least one pawl member that reduces an opening of the retrieving channel to less than a size of the needle assembly and suture, where the pawl member is biased to allow movement of the needle assembly and suture in a first direction and resist movement of the needle assembly and suture in a second direction, where rearward movement of the needle assembly from the retrieving channel causes the paw member to compress and retain the suture within the retrieving channel.

Alternate suture retriever assembly can include structures selected from the group consisting of a set of jaws, a recessed notch, pawl, funnel, catch cloth, magnetic coupling device, finger trap, or other gripping mechanism.

The devices of the present disclosure can include one or more vacuum lumens at the tissue engaging surface for securing tissue thereagainst. Alternately, or in combination, the tissue engaging surface can include a bonding agent for securing tissue thereagainst.

The devices described herein can be combined with various other medical implements to aid in the closure of tissue. For example, the devices can include one or more pledgets that removably positioned on the tissue engaging surface for placement at the opening in tissue.

In another variation, a suture driving assembly for closing an opening in a tissue section can include a first needle assembly having a tissue piercing end distal and being elastically deformable when restrained into a strained state and upon release assumes the curvilinear shape; a suture exterior to the needle assembly and having at least one end front-loaded into a needle lumen of a first tissue piercing portion of the first needle assembly; a main body having a tissue engaging surface at a distal end, at least one constraining channel and at least one retrieving channel each of which having an opening at the tissue engaging surface; where the constraining channel extends through the main body and comprises at least a restraining portion having a profile to maintain the needle assembly into the strained state and a guide segment portion adjacent to the constraining channel opening and having a profile to release needle assembly into the curvilinear shape when advanced therethrough and upon continued advancement the needle assembly exits the opening of the constraining channel in the curvilinear shape; a suture retriever assembly located in the needle retrieving channel and comprising a pawl mechanism, where the pawl mechanism interferes with the front loaded suture and needle assembly when advanced therein, where rearward movement of the front loaded suture and needle assembly causes the pawl to engage the suture to retain the suture within the needle retrieving channel; and an expandable member axially moveable relative to the tissue engaging surface, the expandable member having a first reduced profile and an expanded profile, where in the reduced profile the expandable member can advance through an opening in the tissue section and where the expandable member can be withdrawn toward the tissue supporting face to secure the tissue section therebetween when expanded.

The present disclosure also includes methods positioning a suture in a wall of an organ to close an opening in the wall. In one variation, the method includes placing a main body adjacent to a proximal side of the tissue, where the main body comprises at least one needle assembly coupleable to the suture and within a constraining channel located in the main body, where the needle assembly comprises a tissue piercing end distal to an elongate shaped section, the elongate shape section having a curvilinear shape, the shaped section being elastically deformable into a strained state within the constraining channel, and a suture coupled to the needle assembly, the main body further including a tissue engaging surface; advancing an expandable member through the opening in the organ when the expandable member is in a reduced profile; expanding the expandable member to an expandable profile; positioning the wall of the organ between the main body and the expandable member; advancing the needle assembly from the constraining channel into a guide segment, where the guide segment permits the shaped section of the needle assembly located therein to revert to the curvilinear shape prior to leaving the guide segment and entering the wall of the organ; driving the needle assembly through a proximal side of the wall of the organ, such that the shaped section moves through the curvilinear shape so that the tissue piercing distal end and suture re-enter the main body at a retrieving channel; fully reducing the expandable member into a reduced profile; and withdrawing the main body.

In another variation, the method may further include advancing a plurality of needle assembly pairs, where each needle assembly pair is coupled to an end of a suture and where each needle assembly advances from a respective constraining channel into a respective guide segment, where the guide segment permits the shaped section of the respective needle assembly located therein to revert to the curvilinear shape prior to leaving the respective guide segment and enter the wall of the organ; and where the plurality of needle assemblies move through the curvilinear shape so that the tissue piercing distal end of each needle assembly pair re-enter the main body at a respective retrieving channel.

As described above, the method optionally includes the use of front-loaded sutures. Such sutures allow for securing the suture in the retrieving channel by advancing the needle assembly and suture against a pawl mechanism such that the pawl mechanism compresses the suture to retain the suture while allowing the needle assembly to be withdrawn back into the constraining channel.

The methods can include positioning the wall of the organ between the main body and the expandable member by axially moving the expandable member relative to the tissue engaging surface to capture the wall of the organ therebetween. To further stabilize the device, the method can include partially reducing the expandable member and positioning the partially reduced expandable member into the opening.

In another variation, the methods can include positioning a suture within a tissue of the heart for closing an opening in the heart. Such method can comprise placing a main body adjacent to an exterior surface of the heart, where the main body comprises at least one needle assembly coupled to the suture and within a constraining channel located in the main body, where the needle assembly comprises a tissue piercing end distal to an elongate shaped section, the elongate shape section having a curvilinear shape, the shaped section being elastically deformable into a strained state within the constraining channel, and a suture coupled to the needle assembly, the main body further including a tissue engaging surface; advancing an expandable member through the opening and into the heart when the expandable member is in a reduced profile; expanding the expandable member to an expandable profile; positioning the a portion of the heart between the main body and the expandable member; advancing the needle assembly from the constraining channel into a guide segment, where the guide segment permits the shaped section of the needle assembly located therein to revert to the curvilinear shape prior to leaving the guide segment and entering the wall of the organ; driving the needle assembly through a proximal side of the wall of the organ, such that the shaped section moves through the curvilinear shape so that the tissue piercing distal end and suture re-enter the main body at a retrieving channel; reducing the expandable member into a reduced profile; and withdrawing the main body.

In certain variations, the suture driving assembly can be used to drive a needle without any suture. In such a case, the needle may be left within the tissue (to be removed later, to be absorbed by the native tissue, or for permanent placement.) Accordingly, needle driving assemblies having the same or similar structures disclosed herein are within the scope of this disclosure.

Additional suture driving assemblies are described in U.S. patent application Ser. No. 12/188,430 entitled METHODS AND DEVICES FOR DELIVERING SUTURES TN TISSUE filed on Aug. 8, 2008, the entirety of which is incorporated by reference.

### BRIEF DESCRIPTION OF THE DRAWINGS

FIG. 1 shows an example of a suture driving assembly.

FIG. 2A shows an isometric view of one variation of a needle or needle assembly with a back loaded suture.

FIG. 2B shows a side view of the needle assembly of FIG. 2A.

FIGS. 2C to 2F show variations of front loaded sutures.

FIG. 2G shows a variation of a needle assembly having two curved sections affixed to a single suture.

FIG. 3A illustrates a partial cross sectional view of a distal portion of a suture driving assembly.

FIG. 3B shows the distal portion of a suture driving assembly with the needle assembly advanced through guide segments of constraining channels within the main body.

FIGS. 4A to 4E illustrate a needle assembly advancing at a working surface of a main body of a suture driving assembly.

FIGS. 5A and 5B illustrate the needle assembly and suture respectively after actuation of the assembly.

FIGS. 5C and 5D illustrate an example of a laced suture driven by a needle assembly after passing through tissue about an opening in the tissue.

FIGS. 6A to 6C provide another example of a configuration of a main body having two pairs of constraining and retrieval channels to produce a desired stitch

FIGS. 7A and 7B illustrate examples of needle or suture retrieval devices.

FIGS. 8A to 8D illustrate an example of a suture retrieval device used to retrieve a front-loaded suture.

FIGS. 8E to 8G illustrate another example of a suture retrieval device used to retrieve a front-loaded suture.

FIG. 9A shows another example of a suture driving assembly having a device that extends within a main body of the assembly along with a dilation device.

FIG. 9B illustrates the suture driving assembly of FIG. 9A

FIG. 9C shows an example of a dilation device.

FIG. 9D illustrates a front view of the suture driving assembly of FIG. 9B.

FIG. 9E is a perspective view of a tissue engaging surface of s suture driving assembly where the needle assemblies and sutures are extended from the constraining channels in the main body.

FIG. 9F illustrates an introducer and dilation device that can be advanced through the suture driving assembly of FIG. 9A.

FIGS. 10A to 10F illustrate an example of a suture driving assembly when used to place a suture in an organ. In the present example the suture is used to temporarily secure an access sheath for performing an additional procedure within the heart.

### DESCRIPTION OF THE PREFERRED EMBODIMENT

The above variations are intended to demonstrate the various examples of embodiments of the methods and devices of the invention. It is understood that the embodiments described above may be combined or the aspects of the embodiments may be combined in the claims.

The present invention relates generally to systems and methods for the driving of a needle or suture through or into body tissue (typically, the needle will be affixed to a suture that remains in the tissue) using a catheter, introducer or other minimally invasive means. The methods and devices described herein can be used in any number of medical procedures, including but not limited to, approximating tissue (e.g., bring separated tissue together), ligating tissue (e.g., encircling or tying off), and fixating of tissue (attaching tissue to another structure or different tissue).

As noted herein, the suture driving methods and assemblies described are discussed in relation to vascular wound closure allowing a physician to quickly, easily, and accurately insert a suture immediately following the procedure to prevent excessive blood loss by the patient. In addition the suture driving methods and devices can be used in various other areas (such as cardiology, urology, gynecology, or other vascular surgery applications) to approximate, ligate, or fixate tissue.

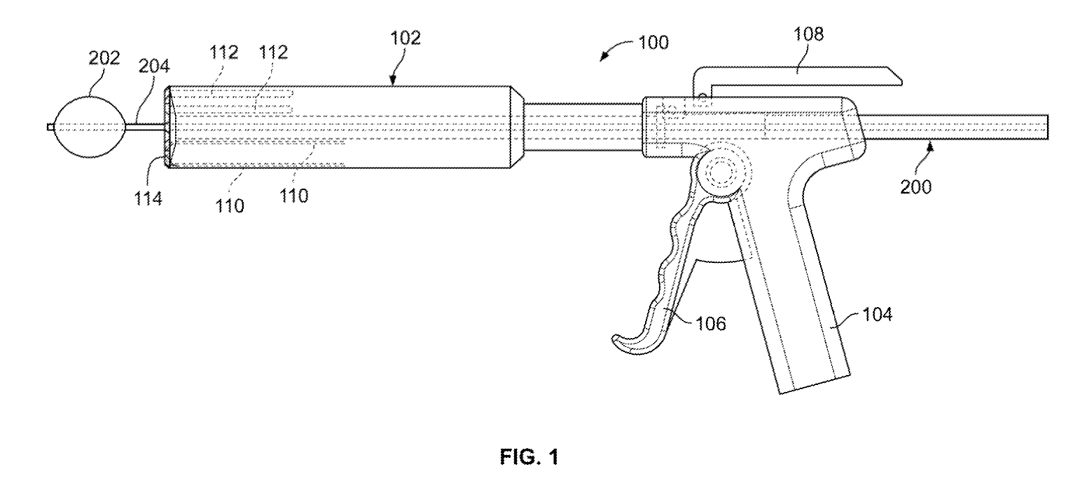

FIG. 1A shows an example of a suture driving assembly 100. In this variation, the suture driving assembly 100 includes a main body 102 coupled to a handle portion 104. The handle portion 104 can include any number of actuating triggers or levers 106, 108 where each lever functions to drive and/or retract the needle assembly to or from tissue. In the illustrated example, the needles are driven through the tissue or “thrown” via a trigger assembly 106. Once the needles are thrown through the tissue and the =sutures are in place, the needle and suture withdraw into the device using a retrieval mechanism 108. Alternatively, variations of the device include passive retrieval assemblies that decouple the suture from the needle assembly once the suture is properly placed through tissue. The needle assembly can then be retracted back into the main body leaving only the suture affixed within one or more retrieval channels 112. Other variations of the device include passive retrieval assemblies where the thrown needle picks up the stored suture and retrieves the suture to properly place it through tissue. In addition, the handle portion 104 can also include ports or couplings for fluid, suction/vacuum, drug delivery, or similar items that require coupling to the device 102.

As discussed below, the main body 102 of the suturing driving assembly 100 includes any number of constraining channels 110 and retrieval channels 112 that open at a tissue engaging surface 114.

The suture driving assembly 100 drives one or more pre-shaped needles (not shown in FIG. 1) through tissue in a manner that allows the pre-shaped needle to revert to its natural state or shape prior to entering tissue. This aspect allows the needle to be first maintained in a pre-deployment shape within a constraining channel 110 and yet deployed from the assembly 100 in the natural state. Such deployment permits the needle (and any attached suture) to pass through tissue in a predetermined path as defined by the natural shape without requiring deformation of the needle.

The suture driving assembly 100 of the present variation can also include an expandable device 200. In the illustrated example, the expandable device 200 comprises an expandable member (in this variation the expandable member is a balloon but the expandable member can be an expandable spline basket, an expandable funnel, a stent-like structure, etc.) 202 affixed to a shaft 204. Additional variations include an expandable member 202 comprising a mechanical basket, a fan shaped element, or any number of expandable structures commonly used in medical applications to secure tissue to a particular surface.

The shaft 204 can be axially moveable relative to the main body 102 so that tissue can be captured between the expandable member (or balloon) 202 of the expandable device 200 and the tissue engaging surface 114. Such movement can occur via a surgeon withdrawing the proximal end of the expandable device 200. Moreover, the variations of the assembly 100 can include various mechanism to lock the position of the expandable portion 202 with respect to the tissue engaging surface 114 so that a surgeon does not need to maintain constant tensile force on the expandable device 200. In addition, the expandable device 200 can include a guide wire lumen to assist in placing the expandable member 202.

As noted above, the tissue engaging surface 114 can also include any number of means to assist with securing tissue 2 against the tissue engaging surface 114 of the main body 102. For example, the tissue engaging surface 114 can include a number of vacuum or suction ports to affix tissue to the surface 144. Moreover, the tissue engaging surface 114 can be rough, channeled, or have other relief contours to move fluid or other substances away from the surface.

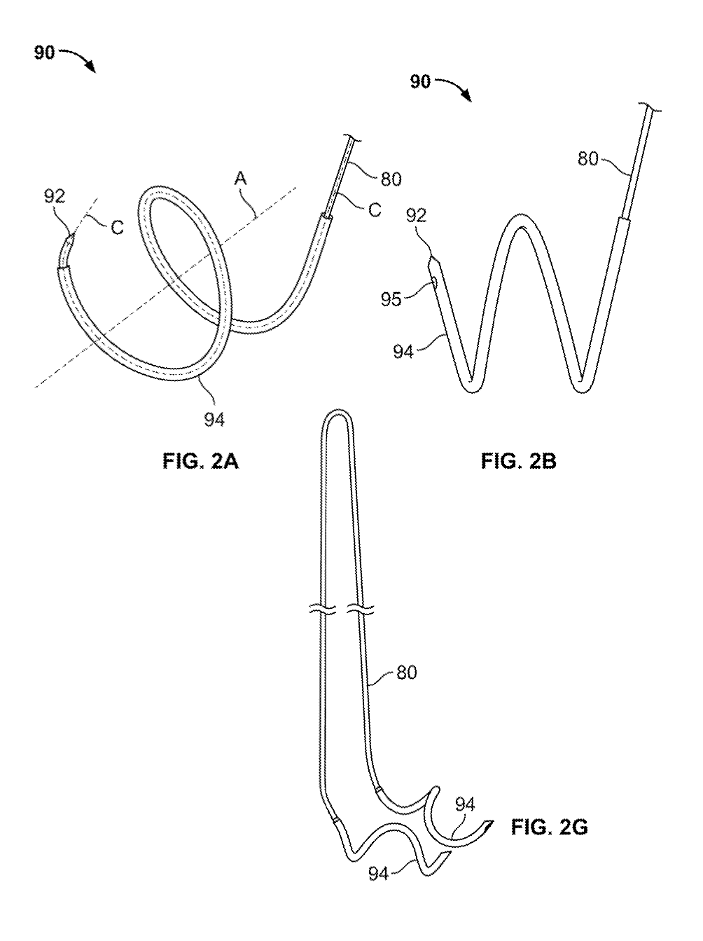

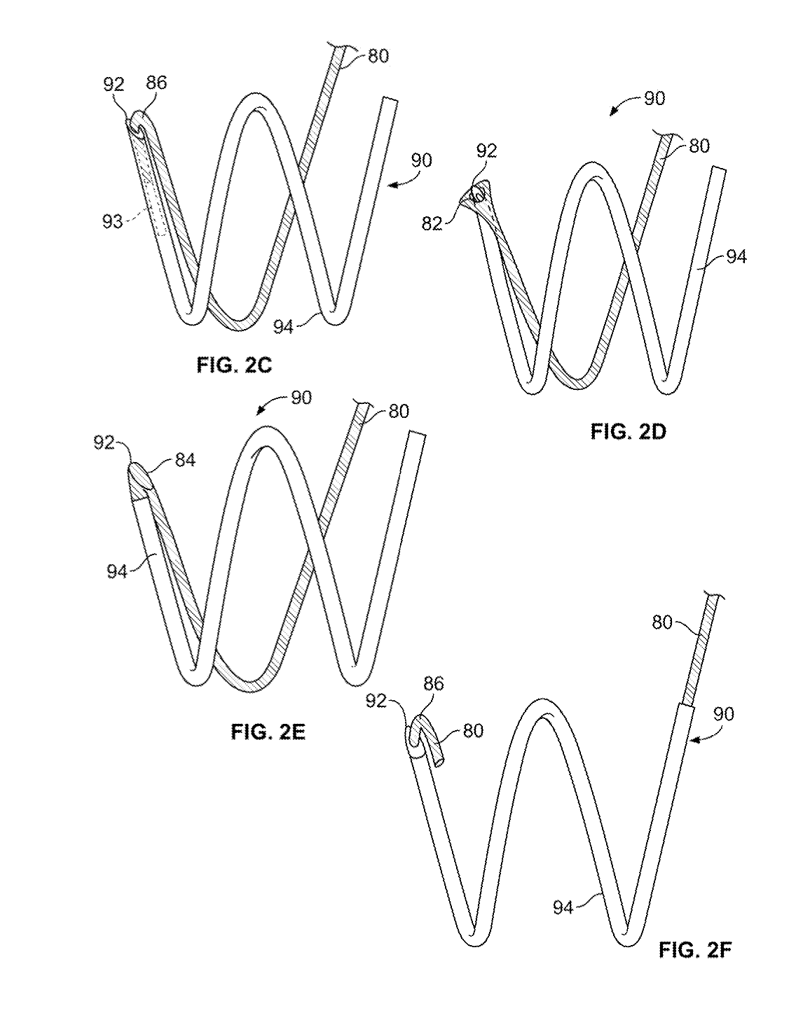

FIGS. 2A and 2B show isometric and side views of one variation of a needle or needle assembly 90. As will be discussed below, the suture driving assembly 100 can include any number of needles or needle pairs depending upon the desired application.

The needle assembly 90 typically comprises a tissue piercing end 92 distal to an elongate shaped section 94. The guide or shaped segment located within the main body allows the elongate shaped section 94 to revert to its natural shape prior to entering tissue as the piercing end 92 and shaped section 94 exits from the main body. The needle assembly 90 also includes a suture 80 coupled thereto. The shaped section 94 of the needles of the present devices includes a curvilinear shape. This shape can be planar (such as a curved needle), or can be three dimensional (as shown by the helix curvilinear shape that wraps about axis A). As noted above, the shaped section 94 of the needle assembly 90 comprises a center line C. In certain variations of the device, the angular bend of the shaped section 94 matches a centerline of the guide or shaped segment located to permit the shaped section 94 to revert to the natural curvilinear shape from a constrained state.

In addition, the shaped section 94 of the needle assembly 90 is elastically deformable into a pre-deployment shape when in the constrained state. Upon release, the shaped section 94 assumes its pre-set curvilinear shape. The needle assembly 90 can also include various features to aid in removal of the needle or suture from the body. For in certain variations of the device, the needle assembly 90 can include a notch, groove, or shoulder adjacent to the tissue piercing tip 92 where the notch 95 increases the ability of a retrieval assembly to withdraw the needle and/or suture. As illustrated, a suture 80 can be “back-fed” into the shaped portion 94. The suture 80 can be glued, crimped, or otherwise affixed to the shaped portion 94.

In some variations, it is desirable to have a needle assembly that does not contain any notch or openings that create areas of increases stress and create a risk of fracture areas. Accordingly, FIGS. 2C to 2E illustrate side views of needle assemblies having a “front-loaded” suture 80. As used herein, a front-loaded suture is one that can be removed from a front or tissue piercing end of the needle assembly. This configuration does not require the needle to be withdrawn through the retrieval channels in main body. Instead, once the needle assembly delivers the suture into the retrieval channel, because the suture is front loaded, it can be decoupled from the needle body at a front or tissue piercing portion. Such front-loaded sutures can be used with variations of the present suture loading device. FIG. 2C illustrates a suture 80 that is front-loaded into a lumen 93 of the needle assembly 90. The bend 86 of the suture 80 as it exits the lumen 93 holds the suture 80 against the shaped needle portion 94 causing the suture to reside within the lumen 93 adjacent to the tissue piercing end 92. Once the suture is advanced into a retrieving channel in the main body, retraction of the shaped section 94 causes the suture 80 to lift or decouple out of the lumen 93. FIG. 2D illustrates a suture 80 as having an opening or aperture 82 that can be hooked onto the tissue piercing end 92. FIG. 2E illustrates a cape 84 that is removably seated on the tissue piercing end 92. Clearly, any means of affixing the suture in a front loaded manner is within the scope of this disclosure. In such front-loaded configurations, rearward movement of the shaped portion 94 when the suture is held such as in the retrieval channel causes the suture 80 to detach from the tissue piercing end 92. The sutures of the front-loaded needle assembly variations extend along an exterior surface of the shaped portion of the needle. As a result, the shaped portion 94 can be retracted or withdrawn while the suture 80 can be advanced into the device and ultimately secured or otherwise tied to accomplish closure of the opening in tissue.

FIG. 2F illustrates another front-loaded suture design where the suture extends through the shaped section 94 of the needle assembly 90. The suture 80 can have an optional bend 86 to secure the suture 80 against the needle assembly 90 during advancement. Alternatively, the needle assembly can have an increased frictional surface in the needle lumen to maintain the suture within the needle lumen when advancing the needle assembly through tissue. The remainder of the suture 80 extends through the shaped section 94 and out of a proximal end of the shaped section 94. Once the tissue piercing end 92 enters a retrieval channel, a suture retrieval device can secure the suture so that the needle assembly 90 can be retracted into a constraining channel.

Although the needles are shown having a helical shape, any number of curvilinear shapes are within the scope of the disclosure. For example, the shapes may be in a single plane or extend to form a 3-dimensional shape. In addition, the curvilinear shapes may have a plurality of curves, a single curve, and/or can be a partial circular shape.

The tissue piercing end and/or curved shaped section 94 can be comprised of a spring steel or other alloy that is set into shape. Alternatively, memory alloys can be employed. Such alloys include superelastic nickel-titanium (NiTi), copper-aluminum-nickel (CuAlNi), copper-zinc-aluminum (CuZnAl), or other shape memory alloys that are well known in the art.

FIG. 2G shows a variation of a needle assembly having two shaped sections 94 affixed to a single suture 80. This particular configuration is useful to produce a “mattress” stitch pattern. Accordingly, the associated suture driving assembly 100 will include multiple constraining channels as well as guide segments.

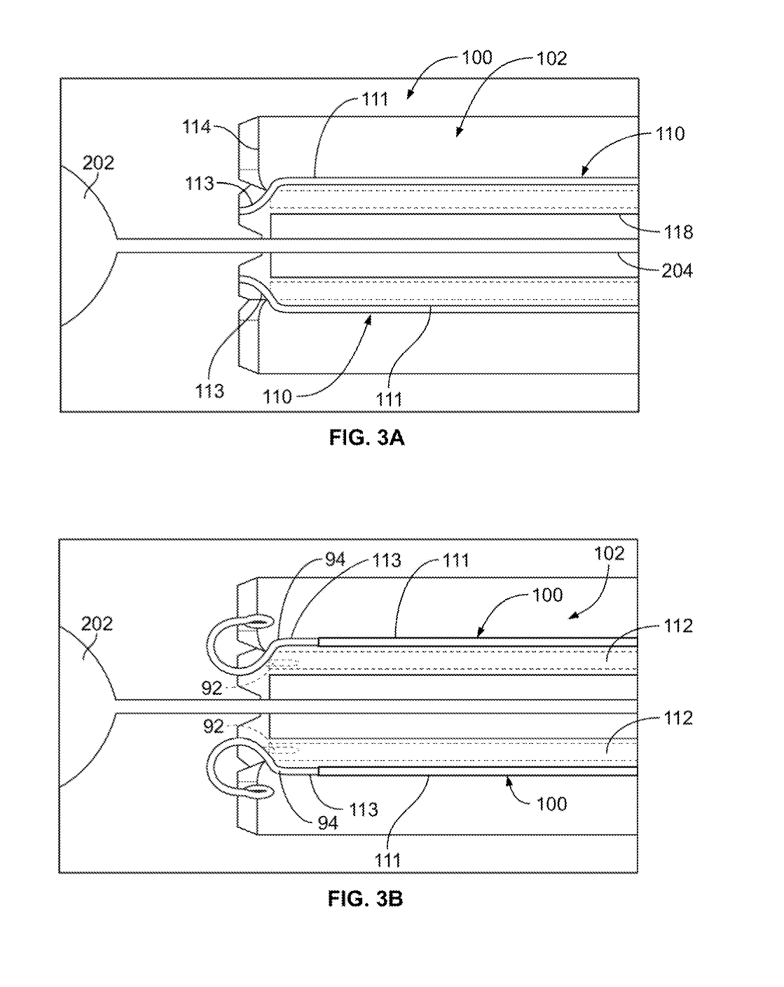

FIG. 3A illustrates a partial cross sectional view of a distal portion of a suture driving assembly 100. As shown, in this variation, the main body 102 includes a main lumen 118 through which the expanding member 202 and shaft 204 can advance. The main body 102 also includes any number of constraining channels 110 that terminate at a tissue engaging surface 114 of the main body 102. The constraining channel 110 can extend fully or partially through the main body 102. A needle assembly, and in certain variations a suture, can be loaded within the constraining channel 110 (though for the sake of illustration the suture and needle assembly are omitted from this figure). The constraining channel 110 comprises at least a restraining portion 111 having a profile to maintain the elongate shaped section into the strained state. In the illustrated variation, the guide portion is linear. However, variations of the device include restraining portions 111 having a variety of shapes.

The restraining portion 111 of the constraining channel 110 transitions into a guide segment 113 that is adjacent to the tissue engaging surface 114. The guide segment comprises a shape or a profile that matches the curvilinear shape of the shaped section of a needle. As a result, as the needle exits the main body, the needle passes through a guide segment having a curvilinear shape that allows the shaped section of the needle assembly to revert to its unconstrained curvilinear shape. Therefore, the needle assembly passes through tissue in its unconstrained state to the retrieval channels as illustrated below. FIG. 313 illustrates the shaped section 94 of a needle assembly as it passes through the guide segment 113 of a constraining channel 110. Once the needle assembly 90 is advanced a sufficient distance, the tissue piercing end 92 enters the retrieval channel 112. As discussed below, the needle assembly 90 can be advanced through the retrieval channel to a retrieval device (not shown) that pulls the needle assembly and suture through the main body. Alternatively, the needle assembly 90 can be retracted back into the constraining channel 110 leaving the suture secured within the retrieval channel 112. Doing so prevents the need of having to retrieve the entire through the main body. Instead, the needle assembly 90 can be withdrawn or retracted into the main body so that only the suture(s) must be retrieved through the main body.

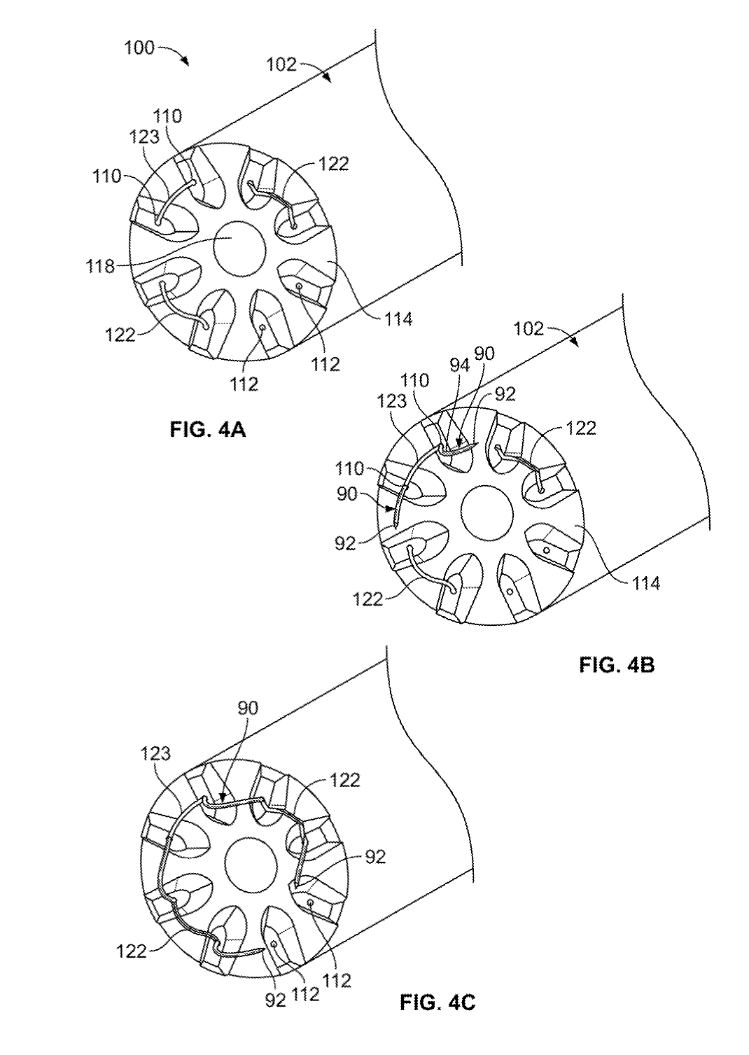

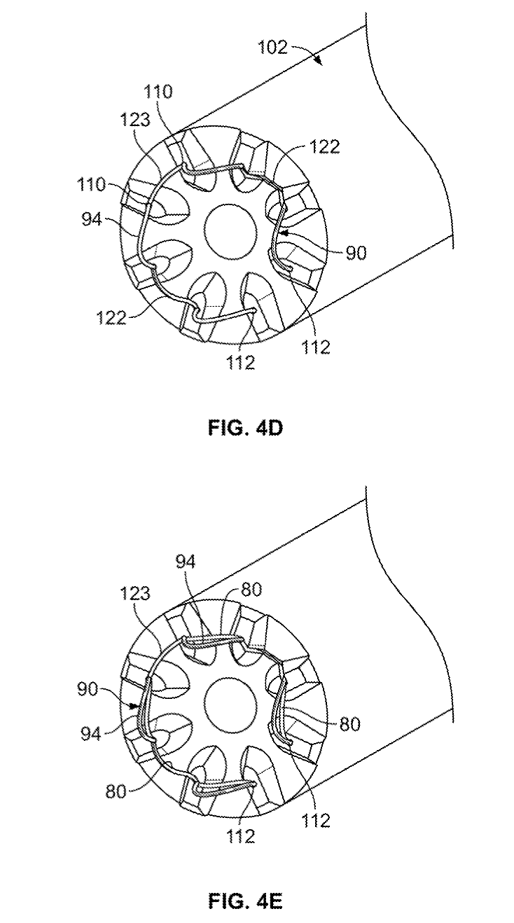

FIG. 4A illustrates a perspective view of a working end of a main body 102 of a suture driving assembly 100. As shown, the main body 102 can include a tissue engaging surface 114 having a number of constraining channels 110 and retrieval channels 112 with respective openings in the tissue engaging surface 114. The number and spacing of the constraining and retrieval channels 110 and 112 will vary depending upon the type of stitch or suture pattern sought. In addition, the sizing of the openings of the constraining and retrieval channels 110 and 112 can also vary.

As discussed above, the tissue engaging surface 114 can be flat, funneled, concave (as shown), or otherwise shaped to ensure proper tissue contact for insertion of a suture. Moreover, the tissue engaging surface 114 can include protrusions 116, channels, or other features to allow fluid to move away from the tissue engaging surface or to better compress the area of tissue in which a needle assembly is to be placed. The main body 102 can also include features such as channels 122 to direct the needle assembly through tissue. Furthermore, the main body 102 can include a main lumen 118 for delivery of an expanding device (not shown) as well as other medical tools/devices. Such a lumen 118 is required in those variations of the device configured for performing procedures within an organ or providing an access path within the organ. FIG. 4A also shows a relief opening 123 between the openings of adjacent constraining channels 110. A relief is typically used in variations of the device having a single suture joined to two needle assemblies 90. The relief opening 123 allows a suture that is joined by two needles to exit the main body when the suture is held within the main body 102. As shown below, variations of the main body 102 can include one or more suture relief openings on a side as well.

FIG. 4B illustrates the main body of FIG. 4A after a needle assembly 90 having two shaped sections 94, is advanced from constraining channels 110 of the main body 102. As discussed herein, the constraining channels 110 can contain a segment adjacent to the opening in the tissue engaging surface 114 that allows the shaped section 94 of the needle assembly 90 to revert to its unconstrained state. This allows the needle assembly to pass through tissue in a manner that is pre-determined by the curvilinear shape of the needle assembly 90. In the illustrated example, the needle assembly 90 is similar to that shown in FIG. 2G. As a result, in the illustrated example, as the shaped sections 94 revert to their unconstrained shape or profile, they orient in a helical curvilinear shape. In order to further direct the needle assembly 90 towards a respective retrieval channel 112, the tissue piercing end 92 of the shaped section 94 enters a guide path or guide channel 122. As shown in FIG. 4C, the guide path 122 deflects the shaped section 94 towards the retrieval channel 114 such that continued advancement of the needle assembly 90. FIG. 4D illustrates the device of FIG. 4C where the tissue piercing ends of the needle assembly 90 are advanced into the retrieval channels 112 in the main body 102.

FIGS. 4A to 4D illustrate one example of a suture advancing device where the suture follows the shaped section 94 of the needle assembly 90. However, as discussed above, variations of the device can include sutures that are front loaded within the needle assembly. FIG. 4E illustrates such an example after the needle assembly 90 is received within the retrieval channel 114 a suture 80 extending along an exterior of the shaped section 94 of the needle assembly 90.

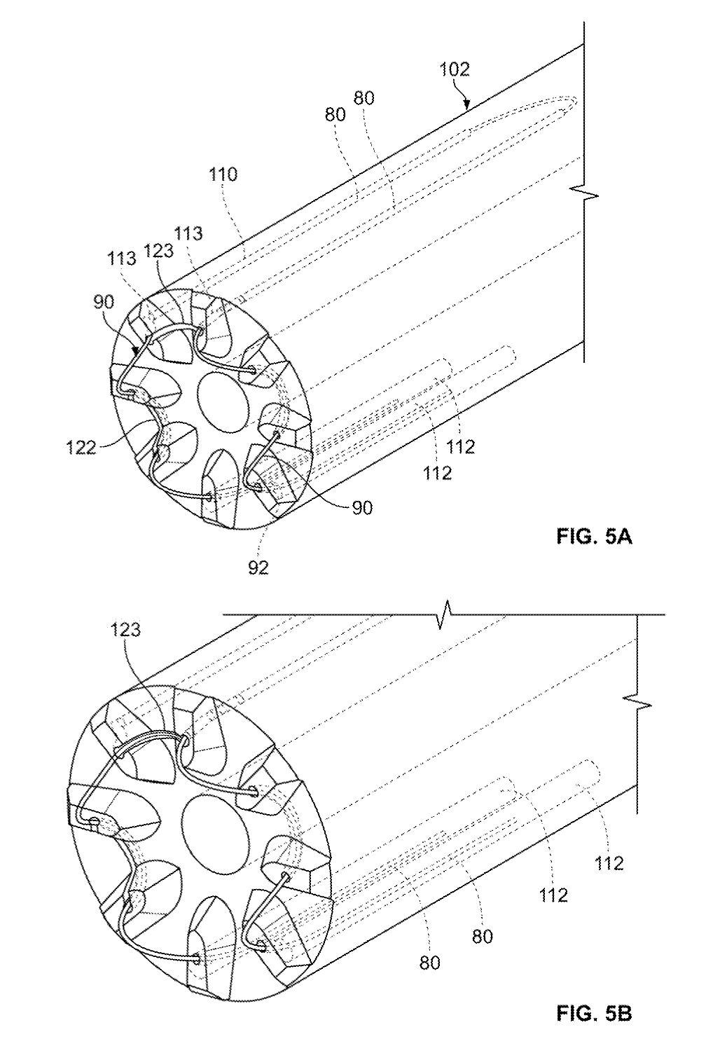

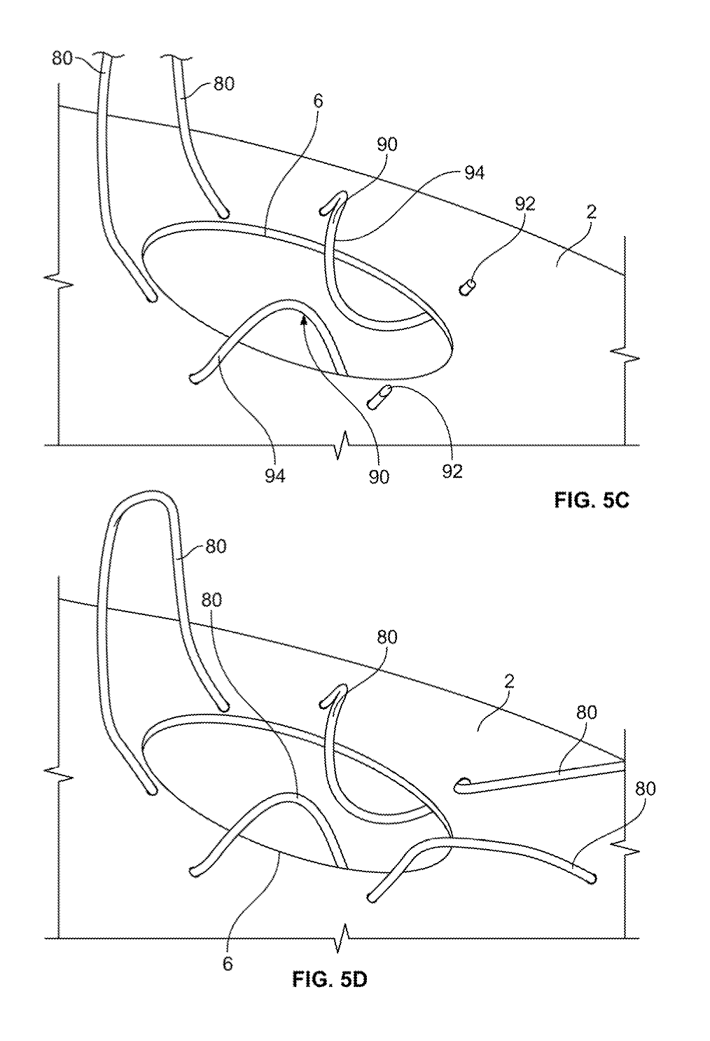

FIGS. 5A and 5B illustrate partially cross-sectional perspective view of a main body 102 having a needle assembly 90 with two shaped sections that is advanced between guide segments 113 of a constraining channels 110 and retrieval channels 112. As shown, the shaped portions of the needle assembly 90 are coupled by a single suture 80. As a result, as the needle assembly 90 leaves the constraining channels 110, the mid section of the suture exits the main body 102 via a suture relief opening 123. The ends of the suture 80 are located within the retrieval channels 112 where any number of mechanisms can be used to withdraw the suture ends.

FIGS. 5C and 5D illustrate respectively, the path of a variation of a needle assembly 90 and suture 80 when advanced in the manner shown in FIGS. 5A and 5B for closing an opening 6 in tissue 2. To clarify the path of the suture and needle assembly, the suture driving assembly is not shown in FIGS. 5C and 5D. As illustrated, the shaped sections 94 of the needle assembly 90 passes through tissue 2 in its unconstrained shape. In the variation shown in FIG. 5C, the needle assembly 90 is coupled to a single suture 80 and pulls the suture 80 through the tissue. FIG. 5D illustrates the state of the tissue 2 after the needle assembly 90 passes from the tissue 2 leaving only the suture 80 remaining in tissue. The resulting laced suture 80 passes through tissue about an opening 6 in the tissue but prior to tightening of the suture 80. Once the suture is “thrown” about the opening 6, the physician can secure the suture to close the opening 6. This particular suture pattern, when tightened, results in a purse string stitch. Clearly, devices within the scope of this disclosure can include any number of tissue receiving openings.

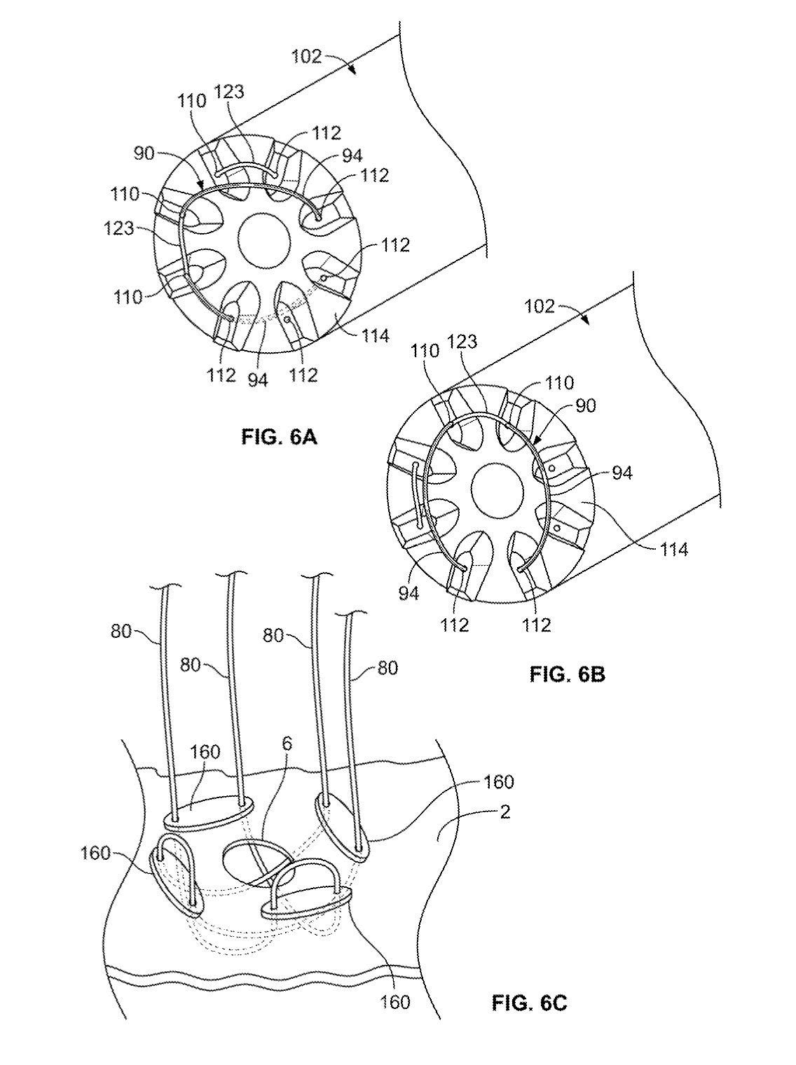

As discussed herein, the configuration of constraining and retrieval channels can be configured in any number of different variations to produce suture patterns as desired. For example, FIGS. 6A to 6C illustrate another such example. Clearly, any number of variations is within the scope of this invention with the illustrated variations depicting some possible variations.

FIGS. 6A and 6B provide another variation such of a main body 102 according to the present disclosure. In this variation, the main body 102 includes two adjacent pairs of constraining channels 110 and two adjacent pairs of retrieval channels 112 having openings in the tissue engaging surface 114. Though each of the openings of the individual pair of constraining channels 110 are joined by a suture relief passage 123, additional variations might not have these passages 123. As noted herein, such passages 123 are required when using a single suture between adjacent shaped sections of a needle assembly. FIG. 6A illustrates a first needle assembly 90 passing between a pair of constraining channels 112 and a pair of retrieval channels 112. FIG. 6B illustrates a second needle assembly 90 passing between the adjacent pair of constraining channels 112 and retrieval channels 112. Although the figures depict the needle assemblies being thrown sequentially, certain variations of the device allow for throwing the first and second needle assemblies at the same time.

FIG. 6C illustrates the sutures 80 depicted in FIGS. 6A and 6B once thrown and when the suture driving apparatus is removed from the tissue 2. As shown, the configuration of FIGS. 6A and 6B produce two perpendicular placed horizontal mattress stitches (one the sutures are properly secured). The illustrated variation also depicts the use of supports or surgical pledgets 160 that can be delivered on the tissue engaging surface of the main body and secured when the needle assembly passes through. Any variation depicted herein can include such pledgets (whether such pledgets are individually spaced about the opening or fully encircle the tissue opening).

Surgical pledgets can comprise biocompatible material (including polyamide, polyethylene, polypropylene, polyethylene terephthalate, polyurethane, polytetrafluoroethylene, various bioresorbable polymers and/or small pieces of autologous tissue. These pledgets are typically used in with the surgical suture to distribute the force of the suture applied on the tissue over a larger area or to aid in steaming the leakage of bodily fluids such as blood that results from penetration of bodily tissue by a suture needle and suture.

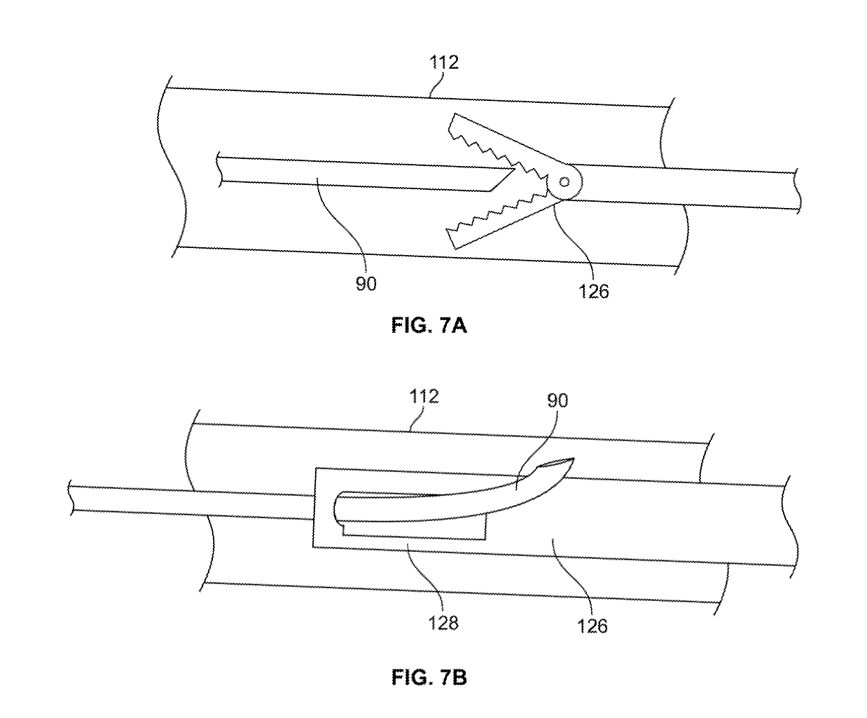

FIGS. 7A to 7B illustrate variations of suture retrieval device 126 that resides within the retrieval channel 112. As shown, as the tissue piercing end 92 of the needle assembly 90 enters the retrieval channel 112 variations of the assembly include a retrieval device 126. In FIG. 7A, the retrieval device 126 shows an example of a clamp or jaw type structure. FIG. 7B shows a retrieval device 126 including a window or slot 128 to capture the tissue piercing tip 92 (or a slot formed in the needle). However, the devices described herein are can include any retrieval device. For example, the retrieval device can comprise a cloth that is penetrated by the needle. The retrieval device 126 can be a finger-trap tubular type of device where tension applied to the device causes compression of the tube allowing for a pulling motion to secure the suture or needle for removal. The retrieval device can be a magnetic coupling device to also aid in removal of the needle or tissue piercing end. In addition, the retrieval devices disclosed in the references discussed in the background section can also be combined with the devices described herein.

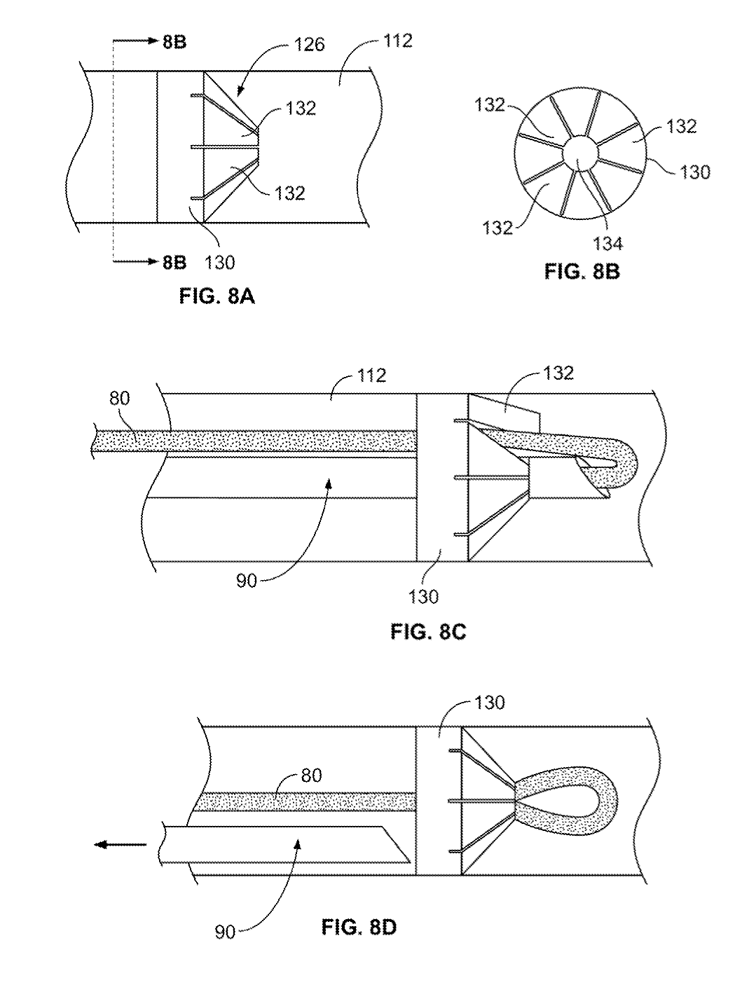

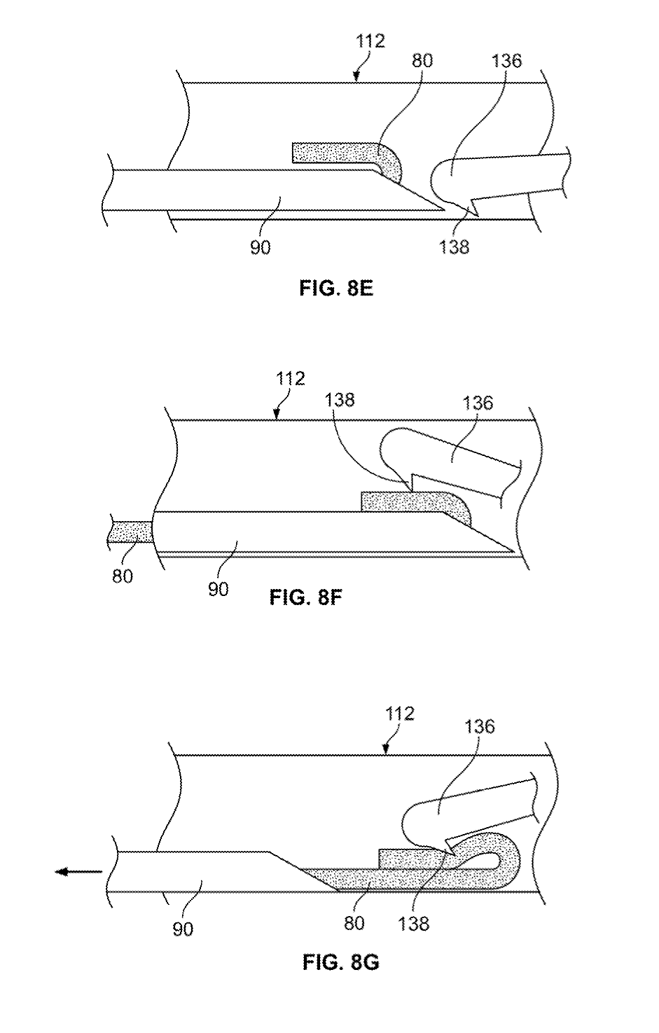

FIG. 8A illustrates another variation of a suture retrieving assembly 126 that comprises a pawl-type mechanism located in the retrieval channel 112. In the illustrated variation, the pawl-type mechanism comprises a slotted funnel 128 where the slots form sections 132 of the funnel that function as pawl-members. As shown by FIG. 8B, from the view taken along line 8B-8B from FIG. 8, the funnel sections 132 form an opening 134 that restricts a diameter of the retrieval channel 112. As shown in FIG. 8C, when a front-loaded suture 80 is loaded into or on a needle assembly 90, the arms 132 of the funnel 130 expand to allow passage of the suture 80 and needle assembly 90 through the funnel 130. However, the funnel members 132 function as a pawl mechanism as they are biased to return to the natural state shown in FIG. 8A. Accordingly, as the needle 90 and suture 80 are retrieved, the arm member (or members) 132 frictionally engage the suture 132 and can compress or bite into the suture. The relatively rigid nature of the needle assembly prevents the funnel members 132 from preventing rearward movement. Accordingly, withdrawing the suture 80 and needle assembly 90 dislodges the front-loaded suture 80 and traps the suture within the funnel 130 as shown in FIG. 8D as the needle assembly 90 is withdrawn. In some variations, the funnel 130 is moveable within the retrieval channel so that the suture 80 can be withdrawn without moving the suture driving assembly. However, in cases where the funnel 130 is stationary, the entire suture driving assembly or the main body alone can be withdrawn to ready the suture for tying about a tissue opening.

Various additional pawl mechanisms are intended to be within the scope of this disclosure, for example, the paw mechanism can comprise a traditional pawl comprising of a hook or tooth located on an arm, where the pawl is biased to engage a suture as it enters the retrieval channel. For example, FIGS. 8E to 8G illustrate a pawl-type mechanism 136 that is biased within a retrieving channel 112 of a device to reduce a size of the channel 112. As illustrated in FIG. 8E, as the front-loaded suture 80 and needle assembly 80 enter the retrieval chamber, the pawl mechanism 136 interferes with the suture 80 and needle assembly 90. Because the pawl-mechanism 136 is spring biased, the suture 80 and needle assembly 90 deflect the pawl-mechanism 136. At this point, as shown in FIG. 8F one or more teeth or protrusions 138 on the pawl-mechanism 136 bite into the deformable suture 80. Once the pawl-mechanism 136 engages the suture 80, the needle assembly 90 can be withdrawn leaving the suture 80 secured within the retrieval channel 112. The pawl-mechanism 136 can then be withdrawn in the channel 112 or the entire device can be withdrawn to pull the secured suture. In addition, the surface of the retrieval channel 112 can have any number of protrusions, hooks, or other features to capture the suture or increase friction against a captured suture.

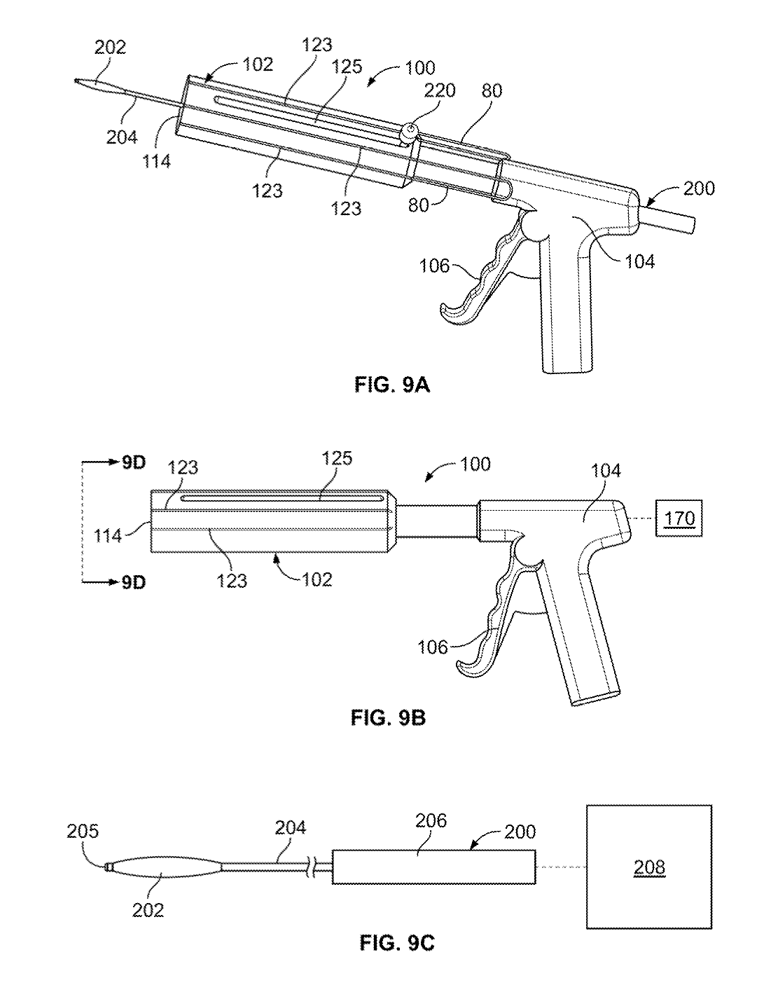

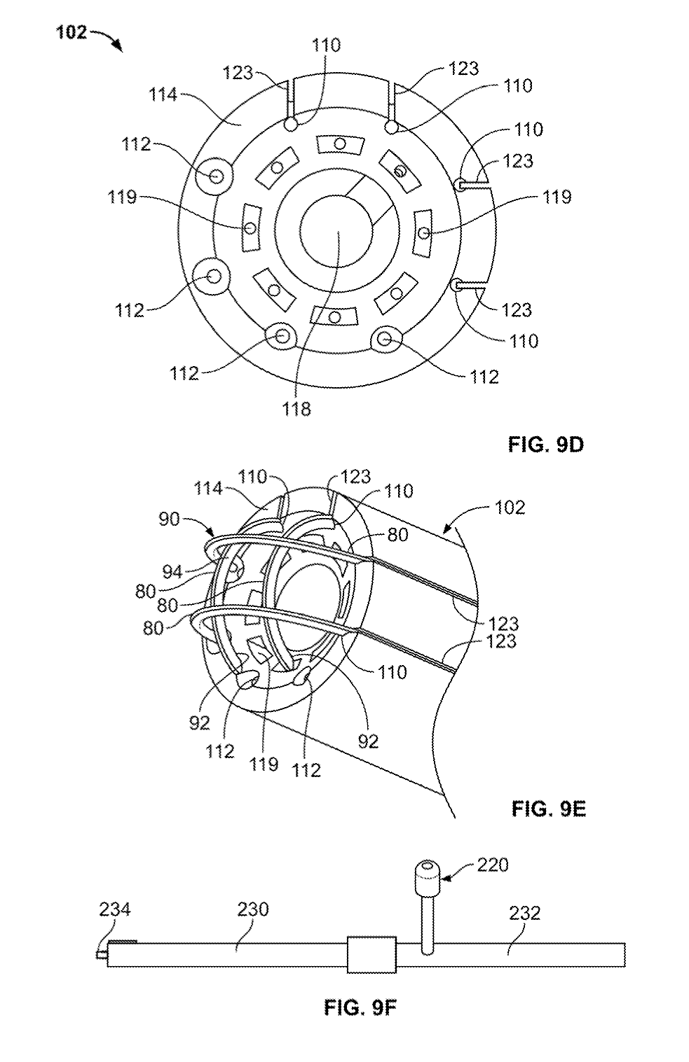

FIG. 9A shows another variation of a suture driving assembly 100 having a expandable device 200 extending through a main lumen. Again, the expandable device 202 can include a balloon 202 or other expandable member affixed to a shaft 204. The expandable device 202 can optionally be stationary within the main lumen or can be moveable relative to the main body 102. The present variation also optionally includes a pin or lever 220 that is moveable within the main body 102. As discussed below, the pin 220 allows advancement of an introducer or other device through the main body. Once the pin 220 is advanced to a desired location, the pin 220 can be removed from the main body 102 to de-couple the main body from the introducer/device located within the main body.

FIG. 9B illustrates the main body 102 of FIG. 9A. As shown, the main body 102 of the present illustration can include a number of suture channels 123 that extend along an exterior surface of the main body 102. As discussed above, such a feature allows a suture to exit the main body when both ends of the suture are joined to one or more needle assemblies that are advanced through tissue. In the illustrated variation and as shown in FIG. 9A, the main sutures 80 can optionally extend through a rear portion of the main body 80. Though not shown, the sutures could be wrapped about spools (not shown) or placed in protective tubing (not shown) rather than remain exposed. Alternatively, the suture channels 123 can extend only through a portion of the main body 102 where the section of suture leaving the suture channel 123 can then be affixed or seated in any portion of the suture driving assembly 100.

FIG. 9B also shows the suture driving assembly as only having a trigger 106. In such variations, the handle portion 104 and trigger 106 rely on a spring based mechanism so that once the trigger 106 is fully actuated, the spring based mechanism releases the trigger 106 and withdraws the needle assembly within the tissue engaging surface 114. As a result, manual retrieval of the needle assembly is not required.

FIG. 9B also illustrates the main body 102 as having a slot 125 to accommodate the pin 220 shown in FIG. 9A. The slot 125 can optionally extend through the proximal and/or distal ends of the main body to allow decoupling.

In those cases where the suture driving assembly 100 relies on a vacuum source 170 to assist in securing tissue against the tissue engaging surface 114, the handle portion 104 or main body 102 can be fluidly coupled to the vacuum source 170 by any conventional means. In addition, the suture driving assembly 100 can also be coupled to any additional fluid supplies to deliver medication, irrigation, or other fluids to the site of the tissue repair.

FIG. 9C shows the expandable device 200 of FIG. 9A. In the present variation, the expandable device 200 can include a shaft 204 having a guide wire lumen 205 extending therethrough. The shaft 204 can have sufficient column strength to allow a surgeon to manipulate a handle 206 at the end of the device 200 to advance the balloon 202 or other expandable member into the tissue being closed. Moreover, in those cases where the expandable device requires a fluid source 208, the handle 206 can include any number of fluid lumens and connectors to fluidly couple the fluid source 208 to the expandable device/balloon 202.

FIG. 9D illustrates the tissue engaging surface 114 as viewed along lines 9D-9D in FIG. 9B. For purposes of illustration, the handle portion and trigger are not shown. FIG. 9D shows the tissue engaging surface 114 of the main body 102 as being tapered or concave with a number of vacuum 119 ports located in the surface 114 and adjacent to both the main lumen 118 and the constraining and retrieval channels 110 and 112. As discussed above, some or all of the ports 119 can be coupled to other fluid delivery sources for irrigation or delivery of other substances.

FIG. 9D also shows two pairs of constraining channels 110 opening onto the tissue engaging surface 114. Each of the constraining channels 110 joins a suture channel 123 as shown in FIGS. 9A and 9B. Accordingly, as the suture advances through the main body, the mid section of the suture can travel outside of the main body 102 along the suture channels 123. The illustration also shows a number of retrieval channels 112 equal to the number of constraining channels 110. In this variation, the retrieval channels 112 have a tapered opening in the tissue engaging surface 114. The suture retrieval devices 126 discussed above can be located within the tapered opening or more distally in the channel 112.

FIG. 9E illustrates the tissue engaging surface 114 of the main body 102 where two pair of needle assemblies 90 are partially deployed from the main body 102. In this variation, the needle assembly pairs 90 are located 90 degrees relative to one another. As shown, the curved section of the needle assemblies 94 comprises a curvilinear shape having a single curve. This variation of the needle assemblies 90 also includes a suture 80 that is front loaded into the needle assembly adjacent to a tissue piercing end 92. FIG. 9E illustrates the needle assemblies 90 just as the tissue piercing ends 92 and front loaded suture 80 are entering the retrieval channels 112. As discussed above, once the needle assemblies 90 enter the retrieval channels 112, a retrieval mechanism (not shown) secures the sutures 80 so that upon retraction of the needle assemblies 90, the sutures 80 remain within the tissue.

FIG. 8F illustrates one example of a device that can be advanced through a main body of a suture driving assembly. In this variation, the device comprises an introducer 230 located on a dilation device 232 having a dilation tip 234 extending from the introducer 230. Advancement of the dilation device 232 and/or introducer 230 can occur via manipulation of the dilation device 232 through a rear end of the suture driving assembly. However, the illustrated variation shows a pin/lever 220 that is irremovably coupled to the dilation device 232 so that the introducer 230 and dilation device 232 can be advanced via movement of the pin 220 as it extends from a slot in the main body as shown in FIG. 9A.

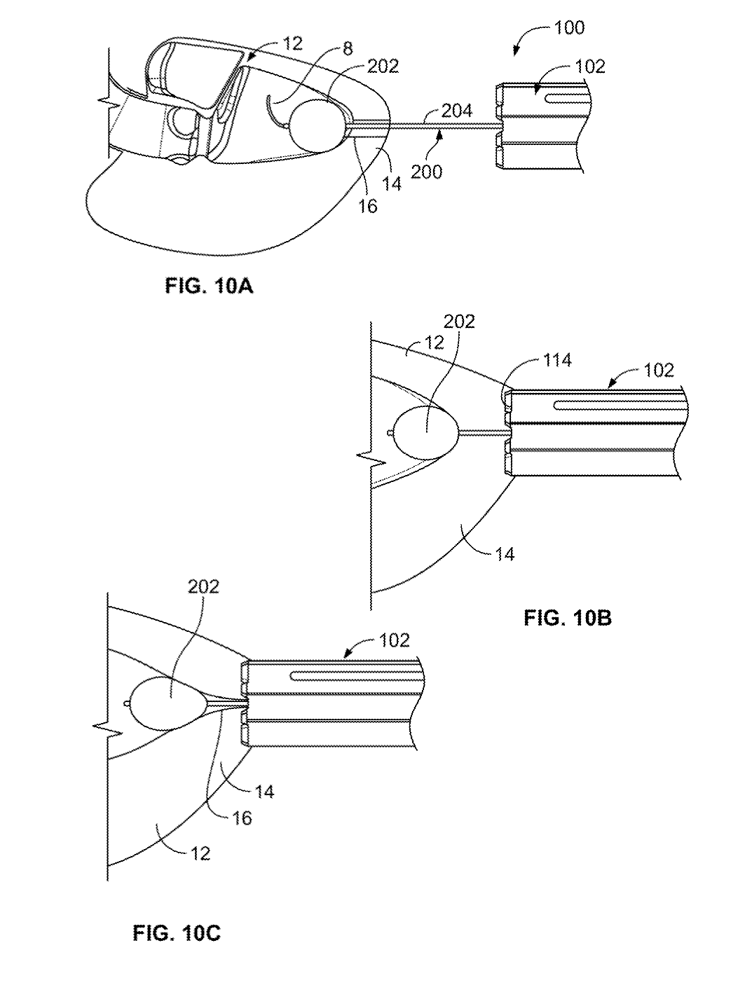

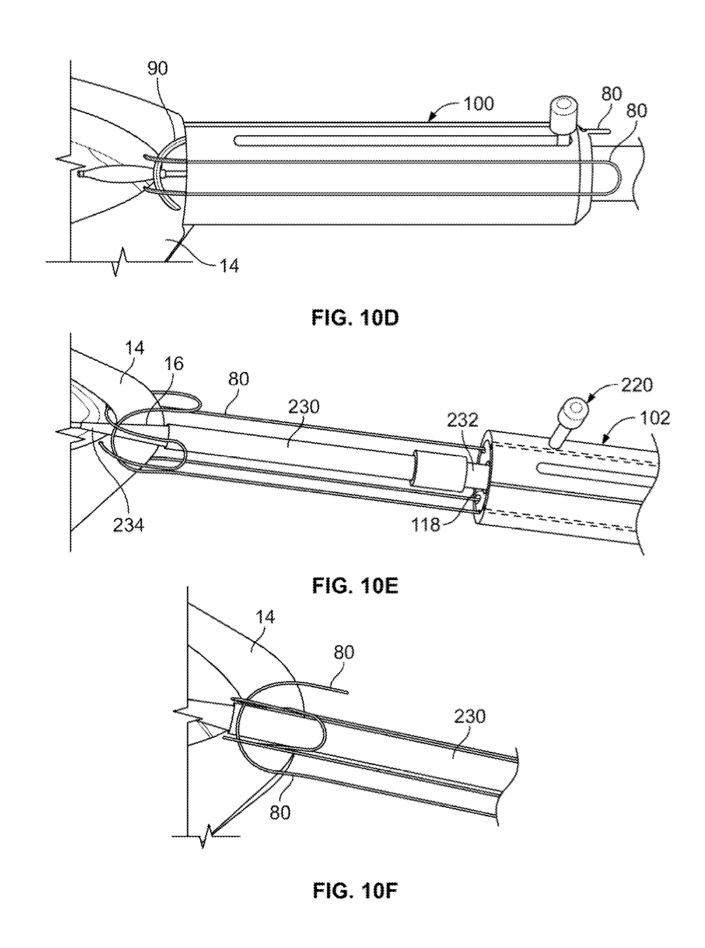

FIGS. 10A to 10F illustrate an example of a procedure for closing an opening in an organ. In this example, the organ comprises an apical portion 14 of a heart 12. However, it is within the scope of this disclosure that the suture driving assembly described above can be used in a variety of situations where closing of a puncture, tear, or opening in tissue is required and in any number of organs. When used in the illustrated apical approach, the suture driving assembly is useful for closing a puncture 16 in the heart's apex 14 after performing a trans-apical valve replacement or repair, when placing a ventricular assist device, or other procedure that would benefit from closing an opening in the heart. As illustrated below, the suture driving device can also be used to deliver additional devices to the tissue site.

FIG. 10A illustrates the heart 12 after a physician tracks a guidewire 8 into an apical portion 14 of the heart where an apical opening 16 allows access to the interior of the heart. Next, the surgeon tracks an expanding device 200 over the guidewire and into the opening at the apical portion 14. In this example the expanding device is a balloon catheter but as noted above, any type of expanding device can be used. Moreover, any traditional technique for tracking a guidewire and catheter can be employed to position the guidewire and expandable device into the heart 12.

Once the physician expands the balloon 202 within the heart 12, the physician can then advance a main body 102 of the suture driving assembly 100 a shaft 204 of the expanding device 200. At this time the surgeon can expand the balloon 202 to minimize dislodging of the assembly from the heart. As shown in FIG. 10B, once the main body 102 of the device 100 engages the apex 14 of the heart 12, the surgeon compresses the apex 14 between the balloon 202 and the tissue engaging surface 114 of the main body. As noted above, the main body can include any number of features to ensure good contact with the tissue. For example, the surgeon can draw suction through ports in the main body to ensure that the apical wall 14 secures to the main body 102.

The physician can optionally fully or partially deflate the balloon 202 (or reduce a diameter of other expandable structures if used). Once reduced, the expandable portion 202 or balloon can be retracted into the apical opening 202 as shown in FIG. 10C. This partial retraction of the balloon into the opening can further stabilize the device to the organ or tissue.

As illustrated in FIG. 10D, once the physician is satisfied with the placement of the device 100, the physician can actuate the device 100 to advance the needle assemblies 100 and throw the sutures 80 through the apical tissue. As noted herein, the needle assemblies can be fired simultaneously or sequentially. In some variations the balloon or expandable member remains inflated during advancement of the needles. In such cases, the balloon/expandable member can be fabricated so that the needle assembly will deflect away from the surface of the expandable member to prevent rupture or trapping of the needles. Moreover, the needle assemblies can fully penetrate the wall of tissue or can remain within the wall. The depth of the throw is typically a function of the type of tissue, the tissue engaging surface, and the design of the needle assembly. In cases such as an apical procedure, the needles may not need to fully penetrate the walls of the organ. For example, the needles in the present example do not reach the balloon but remain within the hearts apical tissue until they return to the retrieval channels.

After placement of the suture the main body 102 can be retracted to expose the suture ends outside the body cavity so that the surgeon can secure the sutures. The balloon can then be deflated and removed. The physician can then place an appropriate port/cannula through the apical puncture 16 and hold the port in place during the procedure by tightening of the stitch. After the procedure is complete, the port/cannula is removed and the purse string suture is drawn tight and secured with a knot or cinch to provide closure of the apical puncture.

FIG. 10E shows an alternative approach, in this variation the surgeon retracts the main body 102 and disengages any vacuum or suction being applied. Next, the surgeon advances a port or introducer sheath 230 the main lumen 118 of the main body 102 and ultimately advanced into the apical opening 16. As noted above, the introducer sheath 230 can be advanced using a pin 220 that slides through an opening in the main body 102. Alternatively, the introducer 230 can be advanced by manipulation of a proximal end of the dilator device 232. The dilator device 232 eases the transition through the apical opening 16 and ensures a seal between the apical opening 16 and introducer sheath 230. The expandable member/balloon can be removed prior to, or during insertion of the introducer sheath 230 within the heart. Alternatively, though not illustrated the balloon can remain in place until the introducer is secured.

As shown in FIG. 10F, once the physician places the introducer sheath 230, the surgeon withdraws the main body 102 to expose the sutures that were previously thrown in the tissue. Removal of the main body allows access to the sutures 80 so that the sutures can be tightened around the dilator 16 and/or introducer sheath 230. As a result, the sutures 230 are temporarily tensioned about the introducer sheath 230 to form a tissue seal around the introducer sheath 230. This temporary fixation allows the physician to create an access path into the heart for performing any appropriate procedures. Once the procedure is completed, the surgeon removes the introducer, and any other remaining devices as necessary while leaving the sutures 80 in place. Once all devices are removed from the opening, the physician secures the sutures 80 to close the apical opening. FIG. 6C provides an example of the suture pattern that will be left in the apical portion of the wall upon removal of the remaining devices.

1. A suture driving assembly for positioning a suture in a tissue section, the assembly comprising:
at least one needle assembly having a tissue piercing end distal to an elongate shaped section, the elongate shaped section having a curvilinear shape, the elongate shaped section being elastically deformable when restrained into a strained state and upon release assumes the curvilinear shape, the suture coupled to the needle assembly;
a main body having a tissue engaging surface at a distal end, at least one constraining channel and at least one retrieving channel each of which having an opening at the tissue engaging surface;
such that when the elongate shaped section of the needle assembly is in the restraining portion, the elongate shaped section is deformed into the strained state and when the elongate shaped section advances through the guide segment portion, the elongate shaped section assumes the curvilinear shape, upon continued advancement the elongated shaped section exits through the opening of constraining channel in the curvilinear shape;
a suture retriever assembly located in the needle retrieving channel;
an expandable member axially moveable relative to the tissue engaging surface, the expandable member having a first reduced profile and an expanded profile, where in the reduced profile the expandable member can advance through an opening in the tissue section and where the expandable member can be withdrawn toward the tissue supporting face to secure the tissue section therebetween.

2. The suture driving assembly of claim 1, where the at least one needle assembly comprises at least two needle assemblies.

3. The suture driving assembly of claim 1, where the constraining channel comprises a first cross-sectional shape and the guide segment has a second cross sectional shape, where the first and second cross-sectional shapes are different, where the second cross sectional shape permits at least a part of the shaped section of the needle assembly entering the guide segment to revert to the curvilinear shape prior to entry into the tissue.

4. (canceled)

5. The suture driving assembly of claim 1, where the expandable member is located on a distal end of a shaft, where the shaft extends through at least a portion of the body where the shaft comprises a guidewire lumen such that the suture driving assembly can be advanced over a guidewire.

6-8. (canceled)

9. The suture driving assembly of claim 1, where the needle assembly comprises a needle lumen extending through at least the tissue piercing end and where the suture is removably nested within the needle lumen and where the suture is exterior to the needle assembly and a first free end of the suture is inserted into the needle lumen at the tissue piercing end.

10. (canceled)

11. The suture driving assembly of claim 9, where a second free end of the suture is coupled to a second needle assembly having a second tissue piercing end distal to a second elongate shaped section, where the second free end of the suture is inserted into a second needle lumen at the second tissue piercing end.

12. The suture driving assembly of claim 1, where the suture is removably front-loaded into the tissue piercing end.

13. The suture driving assembly of claim 12, where the suture retriever assembly comprises at least one pawl member that reduces an opening of the retrieving channel to less than a size of the needle assembly and suture, where the pawl member is biased to allow movement of the needle assembly and suture in a first direction and resist movement of the needle assembly and suture in a second direction, where rearward movement of the needle assembly from the retrieving channel causes the paw member to compress and retain the suture within the retrieving channel.

14-15. (canceled)

16. The suture driving assembly of claim 1, where the shaped section comprises a plurality of curved segments such that advancement of the needle assembly causes the tissue piercing end to penetrate tissue at a plurality of locations.

17. (canceled)

18. The suture driving assembly of claim 1, where the restraining portion maintains the strained state of the shaped section in a substantially linear shape.

19-20. (canceled)

21. The suture driving assembly of claim 1, further comprising at least a second needle assembly comprising a second tissue piercing end distal to a second shaped section having a second curvilinear shape, the second shaped section being elastically deformable into a second strained state and upon release assumes the second curvilinear shape.

22. The suture driving assembly of claim 21, where a second end of the suture is coupled to the second needle assembly.

23. (canceled)

24. The suture driving assembly of claim 1, where the suture retriever assembly comprises a structure selected from the group consisting of a set of jaws, a recessed notch, pawl, funnel, catch cloth, magnetic coupling device, finger trap, or other gripping mechanism.

25. The suture driving assembly of claim 1, where the distal end comprises one or more vacuum lumens for securing tissue thereagainst.

26. The suture driving assembly of claim 1, where the distal end comprises a bonding agent for securing tissue thereagainst.

27. (canceled)

28. A suture driving assembly for closing an opening in a tissue section, the assembly comprising:
a first needle assembly having a tissue piercing end distal and being elastically deformable when restrained into a strained state and upon release assumes the curvilinear shape;
a suture exterior to the needle assembly and having at least one end front-loaded into a needle lumen of a first tissue piercing portion of the first needle assembly;
a main body having a tissue engaging surface at a distal end, at least one constraining channel and at least one retrieving channel each of which having an opening at the tissue engaging surface;
where the constraining channel extends through the main body and comprises at least a restraining portion having a profile to maintain the needle assembly into the strained state and a guide segment portion adjacent to the constraining channel opening and having a profile to release needle assembly into the curvilinear shape when advanced therethrough and upon continued advancement the needle assembly exits the opening of the constraining channel in the curvilinear shape;
a suture retriever assembly located in the needle retrieving channel and comprising a pawl mechanism, where the pawl mechanism interferes with the front loaded suture and needle assembly when advanced therein, where rearward movement of the front loaded suture and needle assembly causes the pawl to engage the suture to retain the suture within the needle retrieving channel; and
an expandable member axially moveable relative to the tissue engaging surface, the expandable member having a first reduced profile and an expanded profile, where in the reduced profile the expandable member can advance through an opening in the tissue section and where the expandable member can be withdrawn toward the tissue supporting face to secure the tissue section therebetween when expanded.

29-44. (canceled)

45. A method for positioning a suture in a wall of an organ to close an opening in the wall, the method comprising:
placing a main body adjacent to a proximal side of the tissue, where the main body comprises at least one needle assembly coupleable to the suture and within a constraining channel located in the main body, where the needle assembly comprises a tissue piercing end distal to an elongate shaped section, the elongate shape section having a curvilinear shape, the shaped section being elastically deformable into a strained state within the constraining channel, and a suture coupled to the needle assembly, the main body further including a tissue engaging surface;
advancing an expandable member through the opening in the organ when the expandable member is in a reduced profile;
expanding the expandable member to an expandable profile;
positioning the wall of the organ between the main body and the expandable member;
advancing the needle assembly from the constraining channel into a guide segment, where the guide segment permits the shaped section of the needle assembly located therein to revert to the curvilinear shape prior to leaving the guide segment and entering the wall of the organ;
driving the needle assembly through a proximal side of the wall of the organ, such that the shaped section moves through the curvilinear shape so that the tissue piercing distal end and suture re-enter the main body at a retrieving channel;
fully reducing the expandable member into a reduced profile; and
withdrawing the main body.

46. The method of claim 45, where advancing the needle assembly comprises advancing a plurality of needle assembly pairs, where each needle assembly pair is coupled to an end of a suture and where each needle assembly advances from a respective constraining channel into a respective guide segment, where the guide segment permits the shaped section of the respective needle assembly located therein to revert to the curvilinear shape prior to leaving the respective guide segment and enter the wall of the organ; and where the plurality of needle assemblies move through the curvilinear shape so that the tissue piercing distal end of each needle assembly pair re-enter the main body at a respective retrieving channel.

47-50. (canceled)

51. The method of claim 45, where the suture is front loaded into the needle assembly such that securing the suture in the retrieving channel comprises advancing the needle assembly and suture against a pawl mechanism such that the pawl mechanism compresses the suture to retain the suture while allowing the needle assembly to be withdrawn back into the constraining channel.

52-54. (canceled)

55. The method of claim 45, where the main body further comprising at least a second needle assembly, coupled to a second end of the suture, where the second needle assembly comprises a second tissue piercing end distal to a second shaped section having a second curvilinear shape, the second shaped section being elastically deformable into a second strained state and upon release assumes the second curvilinear shape.

56-82. (canceled)
